(19) United States
(12) Patent Application Publication
(10) Pub. No.: US 2019/0151229 A1
(43) Pub. Date: May 23, 2019

(54) USE OF AN ESSENTIAL OIL OF EVERLASTING FLOWER IN ORDER TO INCREASE OR RESTORE TACTILE PERCEPTION

(71) Applicant: LABORATOIRES M&L, MANOSQUE (FR)

(72) Inventors: VALERIE CENIZO, MEYRARGUES (FR); GERALDINE LEMAIRE, VALENSOLE (FR); PASCAL PORTES, PUYRICARD (FR)

(21) Appl. No.: 16/313,179

(22) PCT Filed: Jun. 20, 2017
(86) PCT No.: PCT/FR2017/051610

§ 371 Date: Dec. 26, 2018

(30) Foreign Application Priority Data
Jul. 1, 2016 (FR) 1656292

(51) Int. Cl. A61K 8/9789; A61K 8/06; A61Q 19/00; A61K 36/28; A61K 9/00; A61K 9/107; A61P 25/02

(52) U.S. Cl. CPC A61K 8/9789; A61K 8/062; A61K 8/064; A61P 25/02; A61K 36/28; A61K 9/0014; A61K 9/107; A61Q 19/00

(57) ABSTRACT

The invention relates to the use of an essential oil for everlasting flower applied topically to the skin in order to increase or restore tactile perception. The essential oil of everlasting flower can be used to develop the neuron projections of cutaneous sensory nerves in humans.

### SUBJECT OF THE INVENTION

The present invention relates to the use of an essential oil of everlasting flower, applied topically to the skin, for increasing or restoring skin touch perception. The essential oil of everlasting flower makes it possible to develop neuronal projections of cutaneous sensory nerves in human beings.

### BACKGROUND OF THE INVENTION

The skin constitutes a major sensory organ, insofar as it is in direct contact with the outside environment. It continually receives a diversity of thermal, mechanical or chemical stimuli.

Several types of sensory receptors have been identified in the skin. In addition to thermoreceptors, which transmit hot and cold sensitivity, nociceptors, which are pain receptors, and pruriceptors, which are pruritus-specific receptors, various types of mechanical receptors have been identified. The latter, which transmit pressure and vibration sensitivity and fine epicritic or touch sensitivity (which responds to a slight contact with the skin), consist of free endings, of Merkel's disks located in the basal epidermis, of Meissner's corpuscles located in the papillary dermis, of Pacinian corpuscles located in the deep dermis and of Ruffini corpuscles located in the dermis. The distribution of these receptors is different in hairless skin and hairy skin. Their density also varies according to regions; thus, the palm of the hand is particularly well supplied and the facial skin also contains numerous receptors.

These mechanical receptors are innervated by sensory neurons called low-threshold mechanoreceptors (LTMRs) which react to harmless mechanical stimulations. They are categorized as various types, depending on the size of their cell bodies, on the diameter of the axons, on the degree of myelinization and on the axonal conduction speed. Sensory neurons provide information to the central nervous system which in return generates an appropriate response. It is thus possible to recognize objects and to distinguish textures, in particular.

These sensory neurons comprise a cell body from which neurites (axons and dendrites) emerge. The neurites are generally enveloped or in contact with the abovementioned receptors, in particular Merkel's cells.

However, with age and in certain pathological conditions, in particular peripheral neuropathies due for example to diabetes, chemotherapy or to alcohol consumption, or else neurodegenerative diseases such as multiple sclerosis, a decrease in tactile perception, or hypoesthesia, is observed. It is thus considered that tactile deficiencies affect several hundreds of thousands of individuals in France. Nevertheless, the attention to them remains on the fringes.

It is therefore understood that it would be of use to have available a means which makes it possible to restore skin touch perception or to increase it.

It has already been suggested that oral administration (EP 2 438 916; US 2002/040052) or topical administration (KR 2013 0107982) of certain extracts of certain plants could result in neurite extension. However, advantage has been taken of this effect only for treating certain neurodegenerative diseases or for the purpose of improving learning, concentration or memory capacities.

### SUMMARY OF THE INVENTION

The present invention aims to provide another plant extract which makes it possible to promote neurite extension. Another object of the present invention is to provide a plant extract which makes it possible to restore or increase skin touch perception, in particular during the application of a cosmetic product.

In this context, a subject of the invention is the cosmetic, that is to say non-therapeutic, use of an essential oil of everlasting flower, applied topically to the skin, for increasing or restoring skin touch perception, in particular for combating the decrease in skin touch perception due to age.

Another subject of the invention is a cosmetic, that is to say non-therapeutic, process for increasing or restoring skin touch perception, in particular for combating the decrease in skin touch perception due to age, comprising the topical application to the skin of an essential oil of everlasting flower.

A subject of the invention is also an essential oil of everlasting flower, for topical use thereof in a treatment aimed at increasing or restoring skin touch perception, by application to the skin of individuals suffering from peripheral neuropathies, from neurodegenerative diseases or from destruction of nerve endings subsequent to a wound such as a burn.

### DETAILED DESCRIPTION

The present invention uses an essential oil of everlasting flower. Among the everlasting flower species that may be used according to the invention, mention will be made of all those which are of the genus Helichrysum and in particular Helichrysum italicum (or Helichrysum angustifolium D.C.), which is the curry plant, Helichrysum arenarium, which is sandy everlasting, and Helichrysum stoechas or common everlasting flower, without this list being limiting. An essential oil of Helichrysum italicum is preferentially used, regardless of the subspecies in question.

In this description the term “essential oil” is intended to mean the product of hydrodistillation or of steam stripping of the volatile organic compounds present in any part of the everlasting flower or the whole plant, and more particularly in its aerial parts, for example its flowers or flowering heads.

The essential oil of everlasting flower may be included in a cosmetic composition and, in this case, it advantageously represents from 0.001% to 5% by weight and preferentially from 0.01% to 2% by weight, relative to the total weight of the composition used according to the invention.

As emerges from the examples hereinafter, it has been demonstrated that the essential oil of everlasting flower makes it possible to promote neuritogenesis, more specifically to develop the neuronal projections of cutaneous sensory nerves in human beings, such that it can be used to restore or increase skin touch perception. It is possible to take advantage of this property by using the essential oil of everlasting flower as such, or within a cosmetic composition for topical use, under a skin care product. As a variant, the cosmetic composition containing the essential oil of everlasting flower may itself constitute a skin care product. The essential oil of everlasting flower thus makes it possible to increase the tactile sensations felt by the individual on whom this care product is applied, during the application of the product, and in particular to have a better perception of its texture or its glide. The essential oil of everlasting flower or the composition comprising it may be applied to any part of the body and preferably to the skin of the face and/or the hands, which are the most highly innervated areas. It may in particular be applied to the skin of individuals suffering from a loss of tactile sensations, in particular of elderly individuals (typically of menopausal women), or suffering from pathological conditions, in particular from peripheral neuropathies, due in particular to diabetes or to chemotherapy, from neurodegenerative diseases, such as multiple sclerosis, or from a destruction of nerve endings subsequent to a wound such as a burn.

This composition generally contains a physiologically acceptable, in particular cosmetically acceptable, medium, that is to say a medium which does not cause tingling or redness incompatible with cosmetic use. This medium preferably contains an aqueous phase in addition to the oily phase. The composition is preferably in the form of an oil-in-water or water-in-oil emulsion.

The aqueous phase may in particular contain, in addition to the water, at least one constituent chosen from aqueous gelling agents and hydrophilic active agents such as humectants.

The term “aqueous gelling agent” denotes a polymeric compound capable of immobilizing water molecules while becoming hydrated and of thus increasing the viscosity of the aqueous phase. Such a gelling agent may be chosen from: polysaccharides, such as: cellulose and derivatives thereof, modified starches, carrageenans, agar agar, xanthan gum and plant gums such as guar gum or locust bean gum; synthetic polymers and in particular homopolymers of sodium acrylate which are advantageously crosslinked, and also acrylic copolymers, in particular copolymers of sodium acrylate and/or of alkyl(meth)acrylate and/or of hydroxyalkyl (meth)acrylate and/or of (polyethoxy)alkyl (meth)acrylate with at least one other monomer, advantageously 2-acrylamido-2-methylpropanesulfonic acid (AMPS), these copolymers preferably being crosslinked; and mixtures thereof.

For its part, the fatty phase may comprise, in addition to the essential oil of everlasting flower, one or more volatile and/or nonvolatile oils. Examples of volatile oils are branched C₁₀-C₁₃ alkanes, such as isododecane, and linear C₁₀-C₁₃ alkanes. As nonvolatile oils, mention may in particular be made of:

esters of acids and of monoalcohol, chosen from: mono- and polyesters of saturated linear C₂-C₁₀ (preferably C₆-C₁₀) acids and of saturated linear C₁₀-C₁₈ (preferably C₁₀-C₁₄) monoalcohols, mono- and polyesters of saturated linear C₁₀-C₂₀ acids and of branched or unsaturated C₃-C₂₀ (preferably C₃-C₁₀) monoalcohols; mono- and polyesters of branched or unsaturated C₅-C₂₀ acids and of branched or unsaturated C₅-C₂₀ monoalcohols; mono- and polyesters of branched or unsaturated C₅-C₂₀ acids and of linear C₂-C₄ monoalcohols;

C₆-C₁₂ fatty acid triglycerides, such as caprylic and capric acid triglycerides and triheptanoin;

branched and/or unsaturated C₁₀-C₂₀ fatty acids (such as linoleic, lauric and myristic acids);

branched and/or unsaturated C₁₀-C₂₀ fatty alcohols (such as octyldodecanol and oleyl alcohol);

hydrocarbons such as squalane (C₃₀), in particular plant squalane extracted from olive oil, and hemisqualane (C₁₅);

dialkyl carbonates, such as dicaprylyl carbonate and diethylhexyl carbonate;

dialkyl ethers such as dicaprylyl ether; and

mixtures thereof.

Mention may also be made of plant oils which contain one or more of the abovementioned constituents.

As esters of acids and of monoalcohols, mention may in particular be made of monoesters such as the mixture of cocoa caprate and caprylate, ethyl macadamiate, shea butter ethyl ester, isostearyl isostearate, isononyl isononanoate, ethylhexyl isononanoate, hexyl neopentanoate, ethylhexyl neopentanoate, isostearyl neopentanoate, isodecyl neopentanoate, isopropyl myristate, octyldodecyl myristate, isopropyl palmitate, ethylhexyl palmitate, hexyl laurate, isoamyl laurate, cetostearyl nonanoate, propylheptyl capylate, and mixtures thereof. Other esters that may be used are diesters of acids and of monoalcohols such as diisopropyl adipate, diethylhexyl adipate, diisopropyl sebacate and diisoamyl sebacate.

Examples of plant oils are in particular wheat germ, sunflower, argan, hibiscus, coriander, grapeseed, sesame, corn, apricot, castor, shea, avocado, olive and soybean oils, sweet almond oil, and palm, rapeseed, cottonseed, hazelnut, macadamia, jojoba, alfalfa, poppy, pumpkin, sesame, marrow, blackcurrant, evening primrose, lavender, borage, millet, barley, quinoa, rye, safflower, candlenut, passion flower, musk rose, Echium, camelina or camelia oil.

The fatty phase may also comprise at least one fatty-phase structuring agent.

The term “fatty-phase structuring agent” is intended to mean a compound capable of thickening the oils contained in the composition, chosen in particular from waxes, fatty-phase gelling agents, and pasty fatty substances, and also mixtures thereof.

In the context of this description, the term “wax” denotes a fatty substance that is solid at 25° C., with a reversible solid/liquid change of state, having a melting point generally between 30 and 160° C., preferably between 50 and 90° C., as measured by DSC. Examples of waxes are in particular waxes of animal or plant origin, such as beeswax, candelilla wax, carnauba wax or acacia wax; hydrogenated plant oils optionally modified with isostearic acid, in particular hydrogenated rapeseed, soybean, sunflower, jojoba, coconut and castor oils; esters of saturated linear C₁₄-C₃₀ fatty acids and of saturated linear C₁₆-C₃₆ fatty alcohols; linear and saturated C₁₀-C₃₀ acids; linear and saturated C₈-C₃₀ alcohols; and mixtures thereof. These waxes may be in micronized form, that is to say in the form of a powder of which the particles have a number-average size of less than or equal to 50 μm, and in particular ranging from 0.5 to 50 μm, preferably ranging from 1 to 30 μm, or even ranging from 3 to 20 μm, where the “number-average size” corresponds to the dimension given by the statistical particle size distribution to half the population, termed D50.

The term “fatty-phase gelling agents” refers to compounds which modify the rheology of the fatty phase by formation of a three-dimensional network. As compounds of this type, mention may in particular be made of clays (in particular bentonites and hectorites) which are hydrophobically modified, in particular with di stearyldimethylammonium chloride; hydrophobically modified fumed silicas; dextrin palmitate and myristate; polyamides, olefin(s)/styrene copolymers, poly(alkyl acrylate)s; C₁₆-C₂₆ (preferably linear and saturated) fatty acid glycerides such as the compound Nomcort® HKG; cellulose derivatives and mixtures containing same; and mixtures thereof. Some hydrogenated plant oils may also be considered to be fatty-phase gelling agents.

Finally, the pasty fatty substances that can be used as fatty-phase structuring agents are defined as fatty substances with a reversible liquid/solid change of state, exhibiting, in the solid state, an anisotropic crystalline organization and comprising, at a temperature of 23° C., a liquid fraction and a solid fraction. Plant butters are preferably used. Shea butter, cocoa butter and mango butter constitute examples of such pasty fatty substances.

The composition used according to the invention may moreover comprise one or more emulsifiers, fragrances, preservatives, sequestering agents, antioxidants, pH adjusters, UV-screening agents, pulverulent fillers, pigments, dyes, and mixtures thereof.

According to one preferred embodiment, this composition also contains at least one anti-aging active agent, in particular an active agent suitable for preventing and/or treating wrinkles, sagging of the skin and/or the formation of pigment spots, which can in particular be chosen from free-radical scavengers, agents which stimulate keratinocyte and/or fibroblast differentiation and/or proliferation; agents which stimulate the synthesis of glycosaminoglycans and/or of collagen and/or of dermoepidermal anchoring fibrils; agents which prevent the degradation of collagen and/or of glycosaminoglycans and/or of dermoepidermal anchoring fibrils; anti-glycation agents; depigmenting and/or melanogenesis-inhibiting agents; and mixtures thereof. As a variant and/or in addition, the composition according to the invention may comprise at least one active agent with a neuronal effect, such as agents which soothe and/or inhibit neurogenic inflammation; agents which prevent neuro-aging; agents which modulate acetylcholine release by neurons; agents which reduce neuronal exocytosis; and mixtures thereof.

Examples of such anti-aging active agents and/or agents with a neuronal effect are in particular: ascorbic acid, salts thereof, ethers thereof and esters thereof, in particular ascorbyl glucoside; adenosine; ribose; honey extracts; proteins and glycoproteins, extracted in particular from sweet almond; hydrolyzed plant proteins, in particular from rice, from hibiscus seeds or from lupin; polypeptides and pseudodipeptides, such as carcinine hydrochloride, the palmitoyl pentapeptide-4 (Pal-Lys-Thr-Thr-Lys-Ser) and the palmitoyl tripeptide-38 sold in particular by Sederma under the trade names Matrixyl® 3000 and Matrixyl® Synthe′6, respectively, the palmitoyl tripeptide-8 sold by the company Lucas Meyer under the trade reference Nutrazen®, the pentapeptide-18 sold by the company Lipotec under the trade name Leuphasyl® Solution, the sh-decapeptide-9 sold by the company Sandream under the trade name Neoendorphin® and the palmitoyl hexapeptide-52 sold by the company Infinitec under the trade reference X50 Myocept® Powder; silanes, such as methylsilanol mannuronate; arabinoxylans, extracted in particular from ryeflower, and galactoarabinans, in particular from larch; hyaluronic acid and salts thereof; polyphenols, extracted in particular from mimosa; alpha-hydroxy acids, including those extracted from lemon; aqueous extracts of plants such as buckbean, wild pansy, field horsetail, Scottish thistle (Onopordum acanthium), yarrow (Achillea millefolium, contained in particular in the product Neurobiox® from the company BASF), embelia (Embelia concinna, as sold by the company Seppic), Barbary fig (Opuntia ficus indica, sold in particular by Mibelle AG Biochemistry under the trade name AquaCacteen®), sage (Salvia officinalis, sold in particular by Provital Group), Vitex negundo (sold in particular by Laboratoires Expanscience under the trade reference Neurovity®), sweet chestnut, papaya, argan tree, oat, sunflower, daisy, peony or dill; aqueous extracts of algae and in particular of coralline algae, of Jania rubens, of Ungaria pinnatifada, of Alaria esculenta or of Nannochlorosis oculata; essential oils, in particular of myrtle; zinc gluconate and/or copper gluconate; and mixtures thereof.

As a variant or in addition, the composition used according to the invention may comprise at least one tensioning polymer, that is to say polymer capable of tensioning the skin by mechanical action and of thus reducing the appearance of wrinkles and fine lines. It may be a synthetic or natural polymer, in particular a polysaccharide, in particular a marine algal or plankton extract, or a plant gum.

This composition may be in any form suitable for topical application to the skin and in particular in the form of a milk, cream, lotion, gel or mask. It is generally a leave-on composition.

### EXAMPLES

The invention will be understood more clearly on reading the following examples, which are given purely by way of illustration and the purpose of which is not to limit the scope of the invention, defined by the appended claims.

### Example 1

### In Vitro Test on Co-Culture of Human Sensory Neurons and Keratinocytes

1. Materials and Methods

1.1. hiPS Culture and Differentiation

Sensory neurons are derived from iPS cells (induced Pluripotent Stem cells) which are themselves obtained from human fibroblasts. The iPSs were seeded into six-well plates coated with a thin layer of Matrigel® (Corning) in a proportion of 250 000 cells per well in a differentiation medium consisting of DMEM-F12 (Panbiotech) supplemented with 10% of Knockout Serum Replacement (KSR, Life technologies), 5 μM of CHIR 99021 (Tocris), 0.1 μM of retinoic acid (Sigma), 1 μg/ml of EPO (Erythropoietin; Peprotech), a cocktail of central differentiation pathway inhibitors and 1% of PS antibiotics (Penicilin-Streptomycin; Panbiotech). The cells were maintained in culture for 6 days at 37° C. and 5% CO₂. The culture medium was changed every 2 days. Under these culture conditions, the iPSs differentiate into human sensory neurons.

At this stage, the cells were dissociated using accutase (Sigma Aldrich) for 10 minutes at 37° C. The reaction was stopped by adding culture medium. The cell suspension was centrifuged for 5 minutes at 1200 rpm. The cell viability was established by cell counting with trypan blue and the cells were seeded into 96-well plates coated with a thin layer of Matrigel®, in a proportion of 20 000 cells per well, in differentiation medium.

Keratinocytes from an adult donor (Lonza) were pre-amplified, in keratinocyte growth medium (Lonza), over the course of one cycle, before being dissociated by trypsination and frozen. These keratinocytes were thawed and amplified again over the course of one cycle in keratinocyte growth medium.

After 24 hours of adhesion of the neurons in 96-well plates, the keratinocytes were dissociated by trypsination. The cell viability was established by cell counting and the keratinocytes were seeded above the neurons in a proportion of 30 000 cells per well in a culture medium consisting of ⅔ of sensory-neuron maturation medium and ⅓ of keratinocyte growth medium. The maturation medium was composed of DMEM-F12 supplemented with 1% of N₂ (Life technologies), 1% of PS, 10 ng/ml of NGF (Nerve Growth Factor; Sigma Aldrich), 10 ng/ml of BDNF (Brain-Derived Neurotrophic Factor; PanBiotech), 10 ng/ml of NT3 (NeuroTrophin-3; PanBiotech) and 10 ng/ml of GDNF (Glial Derived Neurotrophic Factor; PanBiotech).

1.2. Cytotoxicity of the Essential Oil of Everlasting Flower

In a first culture, after 24 hours of co-culture, the medium was changed for medium consisting of ⅔ of growth factor-free human sensory-neuron maturation medium and ⅓ of keratinocyte growth medium, in the presence or absence of the compound to be tested, at various concentrations ranging from 0.001% to 0.01%. For each concentration, a culture was carried out with six wells.

After 48 hours of incubation, the cells were fixed in a solution of paraformaldehyde (Sigma) at a final concentration of 2% in the culture medium, then the cells were washed twice with a phosphate buffer (PBS, PanBiotech). The nonspecific sites were blocked with a solution of PBS containing 0.1% of saponin (Sigma), 5% of goat serum (GS; PanBiotech) and 1% of BSA (Bovine Serum Albumin; Panbiotech), for 15 minutes at ambient temperature. The cells were subsequently incubated in the presence of an anti β-tubulin mouse monoclonal antibody (Sigma) at 1/400th in PBS containing 5% of GS and 0.1% of saponin, for 2 hours at 4° C. The anti β-tubulin antibody labels the cell bodies and the human sensory neuron projections. This antibody was revealed with a goat anti-mouse IgG Alexa Fluor 488 (Molecular Probe) at 1/400th in a solution of PBS containing 5% of GS, 0.1% of saponin and 1% of BSA for 1 hour at ambient temperature and in the dark. The nuclei were stained with a solution of Hoechst (Sigma), which is a nuclear fluorescent marker, at 1/10000th in the same solution as the secondary antibody.

Twenty photographs per culture well were taken with an automatic microscope (InCell 2000; GE Healthcare) at a ×20 magnification.

1.3. Evaluation of the Effect of the Essential Oil of Everlasting Flower on Neurite Growth

In a second culture, after 24 hours of co-culture, the medium was changed for medium consisting of ⅔ of growth factor-free human sensory-neuron maturation medium and ⅓ of keratinocyte growth medium, in the presence or absence of the compound to be tested, at various concentrations, or of the reference molecule consisting of NGF at 50 ng/ml. NGF or nerve growth factor, which is naturally present in the epithelium, is known to stimulate neurite extension in vitro. A culture was performed with six wells per concentration tested.

The culture was maintained for 6 days and the culture medium containing the compounds of the study was changed after 3 days.

After 6 days of culture, the culture medium was removed and the cells were fixed with a solution of paraformaldehyde at a final concentration of 2% in the culture medium, then the cells were washed twice with PBS. The nonspecific sites were blocked with a solution of PBS containing 0.1% of saponin, 5% of goat serum and 1% of BSA, for 15 minutes at ambient temperature. The cells were subsequently incubated in the presence of an anti β-tubulin mouse monoclonal antibody at 1/400th in PBS containing 5% of GS and 0.1% of saponin, for 2 hours at 4° C. The anti β-tubulin antibody labels the cell bodies and the human sensory neuron projections. This antibody was revealed with a goat anti-mouse IgG Alexa Fluor 488 at 1/400th in a solution of PBS containing 5% of GS, 0.1% of saponin and 1% of BSA, for 1 hour at ambient temperature and in the dark. The nuclei were stained with a solution of Hoechst (Sigma), which was a nuclear fluorescent label, at 1/10000th in the same solution as the secondary antibody.

Twenty photographs per culture well were taken with the automatic microscope (InCell 2000; GE Healthcare) at a ×20 magnification and the cell bodies of the sensory neurons were counted. The length of the projections was also measured and standardized with respect to the number of neuron cell bodies labeled. The results obtained for the conditions for treatment with the compound to be evaluated and the NGF were compared to the control medium.

1.4. Statistical Studies

The statistical studies were carried out using the One-Way ANOVA test.

2. Results

2.1. Cytotoxicity of the Essential Oil of Everlasting Flower

The essential oil of everlasting flower is not toxic to human sensory neurons at the concentrations evaluated.

2.2. Evaluation of the Effect of the Essential Oil of Everlasting Flower on Neurite Growth

The essential oil of everlasting flower at the concentrations evaluated does not cause any modulation of the number of human sensory neurons. This is in agreement with the results obtained during the cytotoxicity study.

NGF at 50 ng/ml causes an increase in the length of the projections of the human sensory neurons (+22.8%, p<0.05). This result validates the experiment.

The essential oil of everlasting flower causes a significant increase in the length of the projections of the sensory neurons at the concentrations of 0.01%, 0.005% and 0.001% (increase respectively of 35.8% p<0.001, 30.8% p<0.001 and 40.3% p<0.001).

3. Conclusion

The essential oil of everlasting flower increases the density of innervation by increasing the length of the projections of the human sensory neurons in co-culture with keratinocytes.

### Example 2

### Cosmetic Composition

The following composition was prepared in a manner that is conventional for those skilled in the art, by mixing the ingredients below in the weight proportions indicated.

| | |
|---|---|
| Nonionic emulsifiers | 6.00% |
| Shea butter | 2.00% |
| Fatty-phase thickeners | 6.50% |
| Oils | 8.25% |
| Adenosine | 0.04% |
| Glycerol | 6.00% |
| Aqueous-phase thickeners | 2.50% |
| Essential oil of everlasting flower | 0.02% |
| Sodium hydroxide | 1.00% |
| Preservatives | qs |
| Fragrance | qs |
| Water | qs 100.00% |

1-8. (canceled)

9. A cosmetic process for increasing or restoring skin touch perception, in particular for combating the decrease in skin touch perception due to age, comprising the topical application to the skin of an essential oil of everlasting flower.

10. The process of claim 9, wherein the everlasting flower is selected from the group consisting of the species Helichrysum italicum, Helichrysum arenarium and Helichrysum stoechas.

11. The process of claim 9, wherein the essential oil is applied to the skin of the face and/or of the hands.

12. The process of claim 9, wherein the essential oil is applied to the skin of elderly individuals.

13. The process of claim 9, wherein the essential oil is included in a composition which is in the form of an oil-in-water or water-in-oil emulsion.

14. The process of claim 13, wherein the composition contains from 0.001% to 5% by weight of essential oil of everlasting flower, relative to the total weight of the composition.

15. The process of claim 14, wherein the composition contains from 0.01% to 2% by weight of essential oil of everlasting flower, relative to the total weight of the composition.

16. The process of claim 10, wherein the everlasting flower is Helichrysum italicum.

17. A method of treatment to increase or restore skin touch perception, by topical application of an essential oil of everlasting flower to the skin of individuals suffering from peripheral neuropathies, from neurodegenerative diseases or from a destruction of nerve endings subsequent to a wound.

18. The method of claim 17, wherein the wound is a burn.
